(19) United States
(12) Patent Application Publication
(10) Pub. No.: US 2019/0211106 A1
(43) Pub. Date: Jul. 11, 2019

(54) NOVEL EGFR VARIANT

(71) Applicant: Exosome Diagnostics, Inc., Waltham, MA (US)

(72) Inventors: Johan Karl Olov SKOG, Charlestown, MA (US); Emily BERGHOFF, Waltham, MA (US); Lori LOGUIDICE, Waltham, MA (US)

(21) Appl. No.: 16/261,241

(22) Filed: Jan. 29, 2019

Related U.S. Application Data
(62) Division of application No. 14/768,275, filed on Aug. 17, 2015, now abandoned.
(60) Provisional application No. 61/765,537, filed on Feb. 15, 2013.

(51) Int. Cl. C07K 16/28; C07K 14/485; C07K 14/71; C12Q 1/6886; C12N 15/113; G01N 33/574

(52) U.S. Cl. CPC C07K 16/2863; C07K 14/485; C07K 14/71; C12Q 1/6886; C12N 15/1138; G01N 33/57492; A61K 47/6849; C07K 2317/76; C12Q 2600/156; C12Q 2600/106; G01N 2333/71; G01N 2800/52; C12N 2310/14

(57) ABSTRACT

The present invention features a novel EGFR variant, EGFRvVI, and methods for detecting the novel EGFR variant. The novel EGFR variant is preferentially expressed in some cancers. Methods for detecting EGFRvVI may aid in the diagnosis, prognosis, and therapeutic assessment of a subject.

### RELATED APPLICATIONS

This application is a divisional application of U.S. application Ser. No. 14/768,275, filed on Aug. 17, 2015, which is a national stage application, filed under 35 U.S.C. § 371, of PCT Application No. PCT/US2014/016536, filed Feb. 14, 2014, which claims benefit of and priority to U.S. Provisional Application No. 61/765,537, filed on Feb. 15, 2013. The contents of each application are hereby incorporated by reference in their entirety.

### INCORPORATION OF SEQUENCE LISTING

The contents of the text file named “EXOS012D01US SeqList.txt, which was created on Jan. 29, 2019 and is 29.7 KB in size, are hereby incorporated by reference in their entirety.

### FIELD OF INVENTION

The present invention relates generally to a novel EGFR variant, to methods for detecting the novel EGFR variant, and to methods for diagnosing and treating cancer.

### BACKGROUND

Growth factors and their receptors play important roles in modulating cell division, proliferation and differentiation. As a result, many growth factor receptors are mutated or aberrantly expressed in cancers, causing deregulation of the growth factor pathways, and resulting in the uncontrolled proliferation that is characteristic of cancer.

Epidermal growth factor receptor (EGFR) is among those growth factor receptors that are frequently mutated, aberrantly expressed, or misregulated in cancers. In certain cancers, such as glioblastoma multiforme (GBM), up to 50% of patients exhibit tumor-specific EGFR variants, such as EGFRvIII. Mutation and amplification of EGFR have also recently been implicated in the oncogenesis and progression of human solid tumors and epithelial tumors and correlated with shorter survival time/rate. Therefore, EGFR has emerged as valuable a biomarker for cancer diagnosis and prognosis.

Importantly, EGFR has also been targeted for anti-cancer therapeutics, with the development of therapies directed towards eliminating the EGFR signal transduction. Two classes of therapies currently exist, antibodies that prevent ligand-mediated EGFR activation and small molecules that inhibit EGFR kinase activity. However, clinical data reveals that not all patients respond to such therapies, which may be a result of tumor-specific expression of different EGFR variants. The EGFRvIII variant lacks a portion of the N-terminal ligand-binding domain. As a result, GBM patients with tumors that express EGFRvIII have been found to be non-responsive to therapies that specifically target the N-terminal ligand-binding domain. Detection of particular EGFR variants has shown utility for predicting responsiveness of a patient to particular EGFR-targeted therapies.

Accordingly, identification of different cancer-associated EGFR variants is useful to provide improved diagnosis, prognosis, stratification and/or therapy guidance of a cancer.

### SUMMARY OF THE INVENTION

The present invention is based on the discovery of a novel EGFR variant, identified in a glioblastoma patient. The present invention addresses the need for cancer biomarkers for more accurate diagnosis of cancer and the predisposition for cancer, as well as biomarkers to guide therapeutic regiments for treatment of cancer. In general, the present invention features a novel EGFR variant, referred to herein as EGFRvVI. The invention also relates to detection of the EGFR variant for the diagnosis, prognosis, and/or therapy guidance of a cancer.

The present invention provides an isolated epithelial growth factor receptor (EGFR) protein comprising an in-frame deletion that starts in exon 3 and ends in exon 7 of wild-type EGFR. In one aspect, the amino acids 90-221 have been deleted. In one aspect, the EGFR protein comprises the amino acid sequence of SEQ ID NO: 5.

The present invention also provides an isolated polynucleotide coding for the epithelial growth factor receptor (EGFR), wherein the nucleotides coding for amino acids 90-221 are deleted. In one aspect, the polynucleotide comprises the nucleotide sequence of SEQ ID NO: 3 or 4.

The present invention also provides an antibody that binds to the EGFR protein of the present invention. Preferably, the antibody has greater affinity for the EGFR variant of the present invention than wild-type EGFR. More preferably, the antibody binds the EGFR variant of the present invention and does not bind to wild-type EGFR.

The present invention also provides a method for diagnosing cancer in a subject comprising analyzing a biological sample for the presence of an EGFR protein or polynucleotide of the present invention, wherein presence of the EGFR protein or polynucleotide indicates the presence of a cancer or a higher predisposition of the subject to develop a cancer. The cancer is a neurologic cancer or an epithelial cancer. In some preferred embodiments, the neurologic cancer is glioblastoma or glioblastoma multiforme. The epithelial cancer is colon cancer, lung cancer, prostate cancer, breast cancer or other solid tumor cancers.

The present invention also provides a method for assessing the responsiveness of a subject to a therapeutic regimen comprising providing a biological sample from a subject and determining the presence, absence, or level of expression of the EGFR protein or polynucleotide of the present invention, wherein the presence, absence, or level of expression of the EGFR protein or polynucleotide indicates the responsiveness of the subject to the therapeutic regimen, wherein the therapeutic regimen comprises an EGFR inhibitor.

The present invention also provides a method for recommending a therapeutic regimen comprising providing a biological sample from a subject, determining the presence of the EGFR protein or polynucleotide of the present invention, wherein the presence of the EGFR protein or polynucleotide rules out a therapeutic regimen comprising an EGFR inhibitor.

In any of the methods described herein, the EGFR inhibitor may be a small molecule, an antibody, a nucleotide, an RNA interfering agent. The EGFR inhibitor inhibits or decreases EGFR activity or mRNA or protein expression. Examples of EGFR inhibitors include, but are not limited to, gefitinib (IRESSA™), erlotinib (TARCEVA®), cetuximab (ERBITUX®), lapatinib (TYKERB®), panitumumab (VECTIBIX®), vandetanib (CAPRELSA®). Other examples of EGFR inhibitors include cancer vaccines that target EGFR deletions, such as Rindopepimut (CDX-110, Celldex Therapeutics). Other inhibitors that can be used with the present invention are PARP1 inhibitors.

In the methods described herein for determining the presence of the EGFR variant, the RNA, DNA or cDNA sequence of the EGFR variant is detected, for example by nucleic acid amplification or quantitative real-time PCR. The amplification product or levels of the EGFR variant can be compared to a reference sample, wherein the reference sample is from a subject does not express EGFRvVI, or expresses wild-type EGFR. The reference sample may be from a subject that does not have cancer. In other embodiments, the reference sample may from the same subject at an earlier timepoint, e.g., before starting a therapy regimen, or before diagnosis of cancer. Changes in the levels or presence of EGFRvVI for a particular subject may aid selection of a therapy or assessment of the efficacy of a therapy.

The present invention also provides a method for targeting a cancer comprising administering to a subject in need thereof an agent that specifically binds to or recognizes the EGFRvVI variant. Preferably, the agent binds to or recognizes the EGFRvVI variant but does not bind to or recognize wild-type EGFR. Thus, the agent specifically recognizes cancer cells. In some aspects, the agent may be covalently linked to a therapeutic agent. For example, the therapeutic agent can be a chemotherapeutic agent, a toxin, a radioisotope, or a nanoparticle.

The present invention further provides a method for treating a cancer or a symptom of a cancer comprising administering to a subject in need thereof an inhibitor of the EGFRvVI variant. The EGFRvVI inhibitor is, for example, a small molecule, an antibody, or an RNA interfering agent. Preferably, the EGFRvVI inhibitor inhibits or decreases EGFR activity or mRNA or protein expression.

Various aspects and embodiments of the invention will now be described in detail. It will be appreciated that modification of the details may be made without departing from the scope of the invention. Further, unless otherwise required by context, singular terms shall include pluralities and plural terms shall include the singular.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representations as to the contents of these documents are based on the information available to the applicants and do not constitute any admission as to the correctness of the dates or contents of these documents.

### BRIEF DESCRIPTION OF THE DRAWINGS

FIG. 1 shows an electropherogram of an RNA sample extracted from 4 ml of cerebrospinal fluid (CSF) sample from a single patient (patient 001). 18S and 28S rRNA peaks are labeled and used to generate the RNA integrity number (RIN).

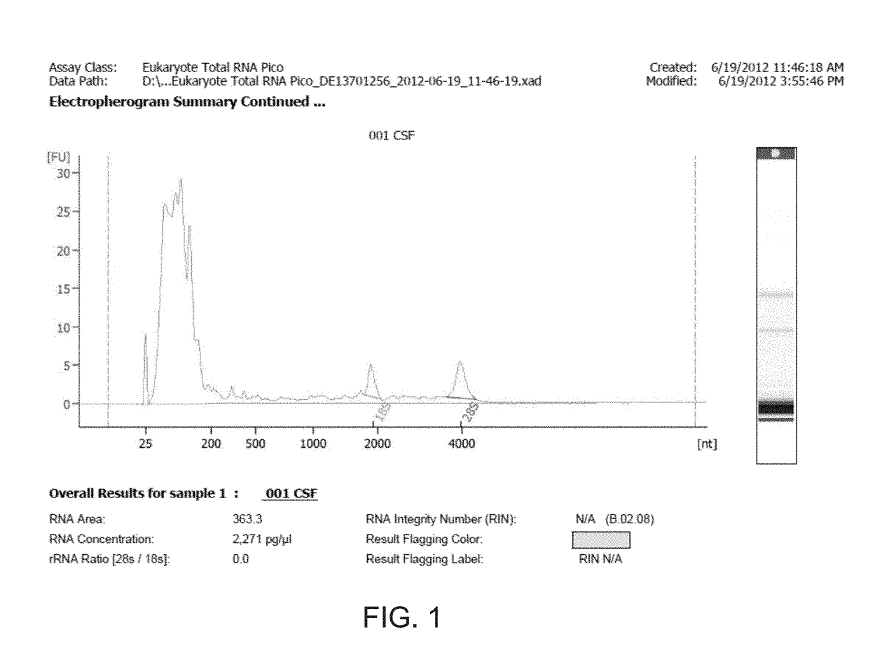

FIG. 2 shows an electropherogram of a typical control sample in the EGFR assay, where no template was used in the QPCR assay. No product is detected.

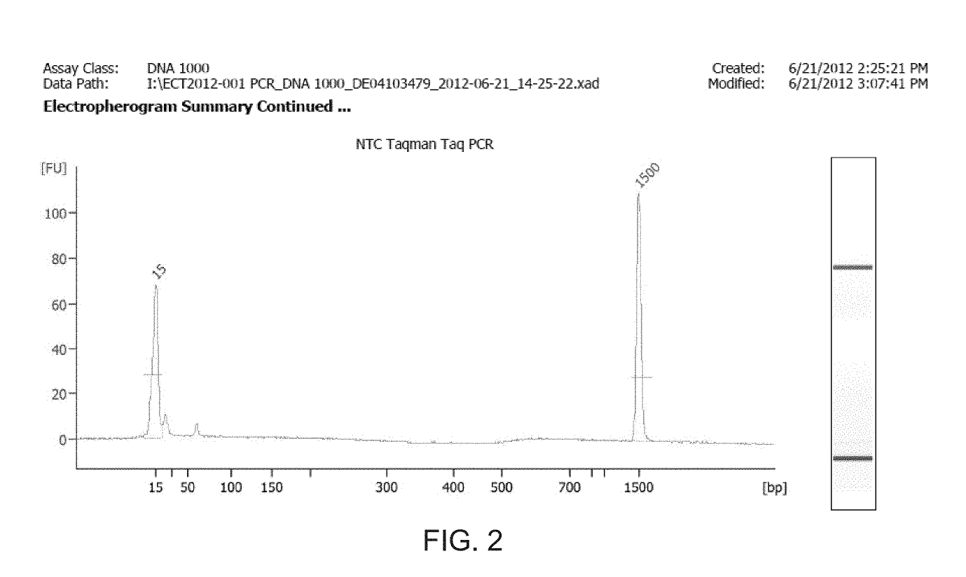

FIG. 3 shows an electropherogram of the RNA extracted from the CSF sample from patient 001 in the EGFR QPCR assay. An amplified product is detected at 397 bp.

FIG. 4 shows a schematic of the deletion at exons 3-7 of the sequenced EGFR nucleotide sequence (partial sequence shown). (A) The gray box designates the homologous region that constitutes the breakpoint in exons 3 and 7 which causes the deletion. (B) This schematic represents select exons in EGFR: exon 1 (italicized); exon 2 (bold); parts of exon 3 (underlined); parts of exon 7 (bold underlined); and exon 8 (bold italicized).

FIG. 5 shows a schematic of the EGFR variants EGFRvIII and EGFRvVI in the context of the full-length EGFR amino acid sequence. The grey box designates the region that is deleted in EGFRvVI, as detected and identified in patient 001. The underlined sequence designates the region that is deleted in EGFRvIII.

### DETAILED DESCRIPTION OF THE INVENTION

The present invention is based on the discovery of a novel variant of the epithelial growth factor receptor (EGFR). Mutations and overexpression of EGFR are known to be associated with a number of cancers, including lung cancer, some epithelial cancers, and glioblastoma multiforme. EGFR mutations, such as the EGFRvIII variant, commonly found in cancers cause constitutive activation of EGFR, resulting in downstream signaling that can cause uncontrolled cell division, a hallmark of cancer. As a result, presence of a mutant form of EGFR can indicate of the presence of cancer or a higher predisposition for cancer. In addition, recent therapeutics have been designed to target EGFR specifically to inhibit EGFR activity. Cancer patients with certain EGFR mutations are often unresponsive to those therapeutics that act through binding or inhibition of the domain or region that is mutated. Thus, EGFR mutant variants are useful for diagnosing whether a subject has cancer or a higher predisposition for cancer, as well as for determining the responsiveness of a cancer patient to a particular therapeutic regimen or for determining the efficacy of a therapeutic regimen.

### EGFR and Cancer

The epidermal growth factor receptor (EGFR) is one of four receptors in the ErbB (also known as HER and Human Epidermal growth factor Receptor) signaling pathway that are present on the cell surface. EGFR is also known as ErbB-1 and HER1. The other members of the HER family include ErbB-2 (HER2/c-neu), ErbB-3 (HER3), and ErbB-4 (HER4).

EGFR is encoded by the c-erbB1 proto-oncogene and has a molecular mass of 170 kDA. A wild-type (WT) EGFR nucleic acid can be encoded by the following human mRNA sequence (GenBank Accession No. BC094761.1) (SEQ ID NO: 1):

GTCCGGGCAGCCCCCGGCGCAGCGCGGCCGCAGCAGCCTCCTCCCCCCGC
ACGGTGTGAGCGCCCGCCGCGGCCGAGGCGGCCGGAGTCCCGAGCTAGCC
CCGGCGGCCGCCGCCGCCCAGACCGGACGACAGGCCACCTCGTCGGCGTC
CGCCCGAGTCCCCGCCTCGCCGCCAACGCCACAACCACCGCGCACGGCCC
CCTGACTCCGTCCAGTATTGATCGGGAGAGCCGGAGCGAGCTCTTCGGGG
AGCAGCGATGCGACCCTCCGGGACGGCCGGGGCAGCGCTCCTGGCGCTGC
TGGCTGCGCTCTGCCCGGCGAGTCGGGCTCTGGAGGAAAAGAAAGTTTGC
CAAGGCACGAGTAACAAGCTCACGCAGTTGGGCACTTTTGAAGATCATTT
TCTCAGCCTCCAGAGGATGTTCAATAACTGTGAGGTGGTCCTTGGGAATT
TGGAAATTACCTATGTGCAGAGGAATTATGATCTTTCCTTCTTAAAGACC
ATCCAGGAGGTGGCTGGTTATGTCCTCATTGCCCTCAACACAGTGGAGCG
AATTCCTTTGGAAAACCTGCAGATCATCAGAGGAAATATGTACTACGAAA
ATTCCTATGCCTTAGCAGTCTTATCTAACTATGATGCAAATAAAACCGGA
CTGAAGGAGCTGCCCATGAGAAATTTACAGGGACAAAAGTGTGATCCAAG
CTGTCCCAATGGGAGCTGCTGGGGTGCAGGAGAGGAGAACTGCCAGAAAC
TGACCAAAATCATCTGTGCCCAGCAGTGCTCCGGGCGCTGCCGTGGCAAG
TCCCCCAGTGACTGCTGCCACAACCAGTGTGCTGCAGGCTGCACAGGCCC
CCGGGAGAGCGACTGCCTGGTCTGCCGCAAATTCCGAGACGAAGCCACGT
GCAAGGACACCTGCCCCCCACTCATGCTCTACAACCCCACCACGTACCAG
ATGGATGTGAACCCCGAGGGCAAATACAGCTTTGGTGCCACCTGCGTGAA
GAAGTGTCCCCGTAATTATGTGGTGACAGATCACGGCTCGTGCGTCCGAG
CCTGTGGGGCCGACAGCTATGAGATGGAGGAAGACGGCGTCCGCAAGTGT
AAGAAGTGCGAAGGGCCTTGCCGCAAAGTGTGTAACGGAATAGGTATTGG
TGAATTTAAAGACTCACTCTCCATAAATGCTACGAATATTAAACACTTCA
AAAACTGCACCTCCATCAGTGGCGATCTCCACATCCTGCCGGTGGCATTT
AGGGGTGACTCCTTCACACATACTCCTCCTCTGGATCCACAGGAACTGGA
TATTCTGAAAACCGTAAAGGAAATCACAGGGTTTTTGCTGATTCAGGCTT
GGCCTGAAAACAGGACGGACCTCCATGCCTTTGAGAACCTAGAAATCATA
CGCGGCAGGACCAAGCAACATGGTCAGTTTTCTCTTGCAGTCGTCAGCCT
GAACATAACATCCTTGGGATTACGCTCCCTCAAGGAGATAAGTGATGGAG
ATGTGATAATTTCAGGAAACAAAAATTTGTGCTATGCAAATACAATAAAC
TGGAAAAAACTGTTTGGGACCTCCGGTCAGAAAACCAAAATTATAAGCAA
CAGAGGTGAAAACAGCTGCAAGGCCACAGGCCAGGTCTGCCATGCCTTGT
GCTCCCCCGAGGGCTGCTGGGGCCCGGAGCCCAGGGACTGCGTCTCTTGC
CGGAATGTCAGCCGAGGCAGGGAATGCGTGGACAAGTGCAACCTTCTGGA
GGGTGAGCCAAGGGAGTTTGTGGAGAACTCTGAGTGCATACAGTGCCACC
CAGAGTGCCTGCCTCAGGCCATGAACATCACCTGCACAGGACGGGGACCA
GACAACTGTATCCAGTGTGCCCACTACATTGACGGCCCCCACTGCGTCAA
GACCTGCCCGGCAGGAGTCATGGGAGAAAACAACACCCTGGTCTGGAAGT
ACGCAGACGCCGGCCATGTGTGCCACCTGTGCCATCCAAACTGCACCTAC
GGATGCACTGGGCCAGGTCTTGAAGGCTGTCCAACGAATGGGCCTAAGAT
CCCGTCCATCGCCACTGGGATGGTGGGGGCCCTCCTCTTGCTGCTGGTGG
TGGCCCTGGGGATCGGCCTCTTCATGCGAAGGCGCCACATCGTTCGGAAG
CGCACGCTGCGGAGGCTGCTGCAGGAGAGGGAGCTTGTGGAGCCTCTTAC
ACCCAGTGGAGAAGCTCCCAACCAAGCTCTCTTGAGGATCTTGAAGGAAA
CTGAATTCAAAAAGATCAAAGTGCTGGGCTCCGGTGCGTTCGGCACGGTG
TATAAGGGACTCTGGATCCCAGAAGGTGAGAAAGTTAAAATTCCCGTCGC
TATCAAGGAATTAAGAGAAGCAACATCTCCGAAAGCCAACAAGGAAATCC
TCGATGAAGCCTACGTGATGGCCAGCGTGGACAACCCCCACGTGTGCCGC
CTGCTGGGCATCTGCCTCACCTCCACCGTGCAGCTCATCACGCAGCTCAT
GCCCTTCGGCTGCCTCCTGGACTATGTCCGGGAACACAAAGACAATATTG
GCTCCCAGTACCTGCTCAACTGGTGTGTGCAGATCGCAAAGGGCATGAAC
TACTTGGAGGACCGTCGCTTGGTGCACCGCGACCTGGCAGCCAGGAACGT
ACTGGTGAAAACACCGCAGCATGTCAAGATCACAGATTTTGGGCTGGCCA
AACTGCTGGGTGCGGAAGAGAAAGAATACCATGCAGAAGGAGGCAAAGTG
CCTATCAAGTGGATGGCATTGGAATCAATTTTACACAGAATCTATACCCA
CCAGAGTGATGTCTGGAGCTACGGGGTGACCGTTTGGGAGTTGATGACCT
TTGGATCCAAGCCATATGACGGAATCCCTGCCAGCGAGATCTCCTCCATC
CTGGAGAAAGGAGAACGCCTCCCTCAGCCACCCATATGTACCATCGATGT
CTACATGATCATGGTCAAGTGCTGGATGATAGACGCAGATAGTCGCCCAA
AGTTCCGTGAGTTGATCATCGAATTCTCCAAAATGGCCCGAGACCCCCAG
CGCTACCTTGTCATTCAGGGGGATGAAAGAATGCATTTGCCAAGTCCTAC
AGACTCCAACTTCTACCGTGCCCTGATGGATGAAGAAGACATGGACGACG
TGGTGGATGCCGACGAGTACCTCATCCCACAGCAGGGCTTCTTCAGCAGC
CCCTCCACGTCACGGACTCCCCTCCTGAGCTCTCTGAGTGCAACCAGCAA
CAATTCCACCGTGGCTTGCATTGATAGAAATGGGCTGCAAAGCTGTCCCA
TCAAGGAAGACAGCTTCTTGCAGCGATACAGCTCAGACCCCACAGGCGCC
TTGACTGAGGACAGCATAGACGACACCTTCCTCCCAGTGCCTGGTGAGTG
GCTTGTCTGGAAACAGTCCTGCTCCTCAACCTCCTCGACCCACTCAGCAG
CAGCCAGTCTCCAGTGTCCAAGCCAGGTGCTCCCTCCAGCATCTCCAGAG
GGGGAAACAGTGGCAGATTTGCAGACACAGTGAAGGGCGTAAGGAGCAGA
TAAACACATGACCGAGCCTGCACAAGCTCTTTGTTGTGTCTGGTTGTTTG
CTGTACCTCTGTTGTAAGAATGAATCTGCAAAATTTCTAGCTTATGAAGC
AAATCACGGACATACACATCTGTATGTGTGAGTGTTCATGATGTGTGTAC
ATCTGTGTATGTGTGTGTGTGTATGTGTGTGTTTGTGACAGATTTGATCC
CTGTTCTCTCTGCTGGCTCTATCTTGACCTGTGAAACGTATATTTAACTA
ATTAAATATTAGTTAATATTAATAAATTTTAAGCTTTATCCAGAAAAAAA
AAAAAAAAA

A wild-type EGFR protein may be encoded by the following human amino acid sequence (GenBank Accession No. AAH94761.1) (SEQ ID NO: 2):

MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQGQKCDPSCP
NGSCWGAGEENCQKLTKIICAQQCSGRCRGKSPSDCCHNQCAAGCTGPRE
SDCLVCRKFRDEATCKDTCPPLMLYNPTTYQMDVNPEGKYSFGATCVKKC
PRNYVVTDHGSCVRACGADSYEMEEDGVRKCKKCEGPCRKVCNGIGIGEF
KDSLSINATNIKHFKNCTSISGDLHILPVAFRGDSFTHTPPLDPQELDIL
KTVKEITGFLLIQAWPENRIDLHAFENLEIIRGRTKQHGQFSLAVVSLNI
TSLGLRSLKEISDGDVIISGNKNLCYANTINWKKLFGTSGQKTKIISNRG
ENSCKATGQVCHALCSPEGCWGPEPRDCVSCRNVSRGRECVDKCNLLEGE
PREFVENSECIQCHPECLPQAMNITCTGRGPDNCIQCAHYIDGPHCVKTC
PAGVMGENNTLVWKYADAGHVCHLCHPNCTYGCTGPGLEGCPTNGPKIPS
IATGMVGALLLLLVVALGIGLFMRRRHIVRKRTLRRLLQERELVEPLIPS
GEAPNQALLRILKETEFKKIKVLGSGAFGTVYKGLWIPEGEKVKIPVAIK
ELREATSPKANKEILDEAYVMASVDNPHVCRLLGICLTSTVQLITQLMPF
GCLLDYVREHKDNIGSQYLLNWCVQIAKGMNYLEDRRLVHRDLAARNVLV
KTPQHVKITDFGLAKLLGAEEKEYHAEGGKVPIKWMALESILHRIYTHQS
DVWSYGVTVWELMTFGSKPYDGIPASEISSILEKGERLPQPPICTIDVYM
IMVKCWMIDADSRPKFRELIIEFSKMARDPQRYLVIQGDERMHLPSPIDS
NFYRALMDEEDMDDVVDADEYLIPQQGFFSSPSTSRTPLLSSLSATSNNS
TVACIDRNGLQSCPIKEDSFLQRYSSDPTGALTEDSIDDTFLPVPGEWLV
WKQSCSSTSSTHSAAASLQCPSQVLPPASPEGETVADLQTQ

Multiple precursor isoforms and alternatively spliced transcript variants that encode different EGFR variants have been identified. The nucleic acid and polypeptide sequences of multiple precursor isoforms of EGFR are known in the art: for example, isoform A (GenBank Accession Number NM_005228.3 and NP_005219.2), isoform B (NM_201282.1 and NP_958439.1), isoform C (NM_201283.1 and NP_958440.1) and isoform D (NM_201284.1 and NP_958441.1). The present invention features an EGFR variant that has a deletion that begins within exon 3 to within exon 7, thereby deleting part of exon 3, all of exons 4-6, and part of exon 7. The deletion is preferably a deletion of amino acids 90-221 of wild-type EGFR.

EGFR and other HER receptors are receptor tyrosine kinases, and are activated though binding of its specific ligands. EGFR ligands are known in the art and include growth factors such as EGF (epidermal growth factor), TGF-α (transforming growth factor alpha), HB-EGF (heparin-binding EGF-like growth factor), amphiregulin (AR), betacellulin (BTC), epigen and epiregulin (EPR). Upon binding of one of its ligands, EGFR is activated to form a homodimer with another EGFR monomer. In some instances, EGFR may also pair with another member of the ErbB receptor family to form an active heterodimer. EGFR dimerization stimulates its intrinsic intracellular protein tyrosine kinase activity, resulting in autophosphoryaltion of several tyrosine (Y) residues in the C-terminal domain of EGFR. The tyrosines that can be phosphorylated include Y992, Y1045, Y1069, Y1148 and Y1173. Tyrosine autophosphorylation activates downstream signaling by several other proteins that are associated with the phosphorylated tyrosines of EGFR through phosphor-tyrosine-binding SH2 domains. Such downstream signaling can activate several signal transduction cascades, principally the MAPK, Akt and JNK pathways. These signaling cascades are known to play essential roles in DNA synthesis, cell proliferation and cell growth, as well as modulation of cellular phenotypes, such as cell migration and adhesion.

EGFR and other components of the HER signaling pathway interact in a complex and tightly regulated manner to regulate cell growth. Alterations in the amount or activity of HER family members, for example, EGFR, may cause or support the inappropriate cell growth that leads to proliferation, migration, and survival of cancer cells. Because the signaling pathway works as a cascade that amplifies the growth signal at each step, small changes in the amount or activity of EGFR may significantly drive the development, or progression, of cancer by promoting cell growth and metastasis, (e.g., cell migration) and inhibiting apoptosis (programmed cell death).

Mutations and overexpression of EGFR have been implicated as important factors in the proliferation of malignancies and have been utilized as markers of poor prognosis for certain cancers. EGFR was the first cell surface glycoprotein identified to be amplified and rearranged in glioblastoma multiforme (GBM) and to act oncogenically to stimulate the growth and spread of cancer cells. EGFR has also been shown to play a role in oncogenesis and progression of other cancers, such as glioblastoma, astrocytoma, meningioma, head and neck squamous cell cancer, melanoma, cervical cancer, renal cell cancer, lung cancer, prostate cancer, bladder cancer, colorectal cancer, pancreatic cancer and breast cancer.

### Identification of a Novel EGFR Variant

Many different variants, also referred to herein as mutants, of EGFR have been identified, and many of these EGFR variants have been implicated in the initiation or progression of certain cancers. Mutations of EGFR are commonly small deletions in the N or C terminus. EGFRvIII, which is an in-frame deletion corresponding to exons 2-7 in the mRNA, is presently known as the most common EGFR variant and was first identified in glioblastoma multiforme (GBM).

EGFR variants known in the art include: EGFRvI (N-terminal truncation), EGFRvII (deletion of exons 14-15), EGFRvIII (deletion of exons 2-7), EGFRvIII/Δ12-13 (deletion of exons 2-7 and exons 12-13), EGFRvIV (deletion of exons 25-27), EGFRvV (C-terminal truncation, EGFR.TDM/2-7 (tandem duplications of exons 2-7), EGFR.TDM/18-25 (tandem duplications of exons 18-25) and EGFR.TDM/18-26 (tandem duplications of exons 18-26).

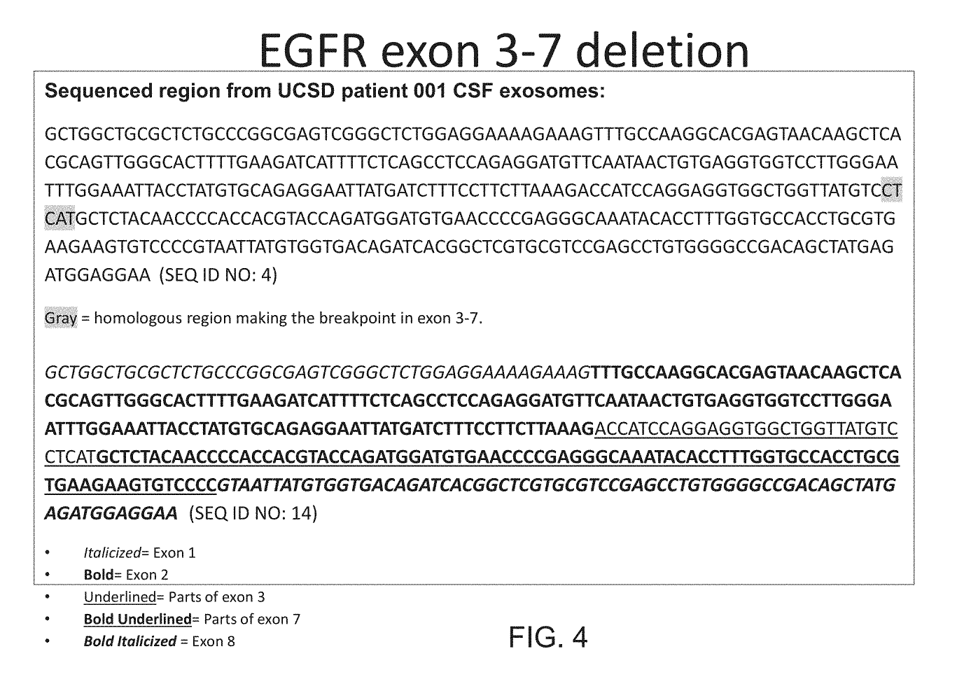

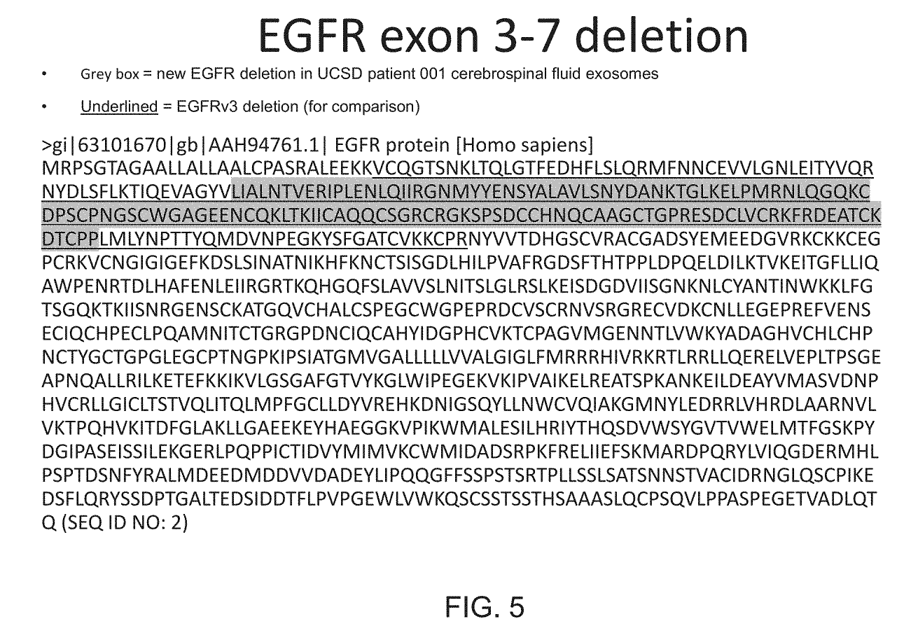

The present invention features a novel EGFR variant, referred to herein as EGFRvVI. The novel EGFR variant was identified as described in Example 1. EGFRvVI contains an in-frame deletion that corresponds to part of exon 3 through part of exon 7. Specifically, the deletion begins within exon 3 and ends within exon 7. Preferably, the deletion begins one third into exon 3 and ends halfway into exon 7. The homologous region constituting the breakpoint within exon 3 and exon 7 that causes the deletion is depicted in FIG. 4. Preferably, the deletion is from amino acids 90-221 of the wild-type EGFR (e.g., SEQ ID NO: 2), and results in an EGFR variant that is 959 amino acids long (e.g., SEQ ID NO: 5), as shown in FIG. 5. Preferably, the deletion is 10730 base pairs (bp) long. In some aspects, the variant may have 1, 2, 3, 4, 5, 10, 15 or 20 additional amino acids deleted from the sequences N-terminal to amino acid at position 90 or C-terminal to the amino acid at position 221 of the wild-type EGFR. Conversely, the variant may have a 1, 2, 3, 4, 5, 10, 15, or 20 additional amino acids C-terminal to amino acid position 90 or N-terminal to the amino acid at position 221, wherein the additional amino acids are identical to the amino acids at the corresponding positions of the wild-type EGFR. The deletions described herein may be present in any of the splice variants or isoforms of EGFR.

In one aspect, the EGFR variant comprises a nucleotide sequence greater than 60%, 65%, 70%, 75%. 80%, 85%, 90%, 91%, 92%, 93%, 94%, 95%, 96%, 97%, 98%, or 99% identity to the nucleotide sequence of the EGFR variant, e.g., SEQ ID NO: 1. In another aspect, the EGFR variant comprises an amino acid sequence greater than 60%, 65%, 70%, 75%. 80%, 85%, 90%, 91%, 92%, 93%, 94%, 95%, 96%, 97%, 98%, or 99% identity to the amino acid sequence of the EGFR variant, e.g., SEQ ID NO: 5.

The term “% identity,” in the context of two or more nucleotide or amino acid sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For example, % identity is relative to the entire length of the coding regions of the sequences being compared.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using search algorithms such as BLAST and PSI-BLAST (Altschul et al., 1990, J Mol Biol 215:3, 403-410; Altschul et al., 1997, Nucleic Acids Res 25:17, 3389-402).

The present invention features a novel EGFR variant, EGFRvVI, comprising the nucleotide sequence shown below. The underlined sequence indicates the homologous region that constitutes the breakpoint within exons 3 and 7.

(SEQ ID NO: 3)
GTCCGGGCAGCCCCCGGCGCAGCGCGGCCGCAGCAGCCTCCTCCCCCCGC
ACGGTGTGAGCGCCCGCCGCGGCCGAGGCGGCCGGAGTCCCGAGCTAGCC
CCGGCGGCCGCCGCCGCCCAGACCGGACGACAGGCCACCTCGTCGGCGTC
CGCCCGAGTCCCCGCCTCGCCGCCAACGCCACAACCACCGCGCACGGCCC
CCTGACTCCGTCCAGTATTGATCGGGAGAGCCGGAGCGAGCTCTTCGGGG
AGCAGCGATGCGACCCTCCGGGACGGCCGGGGCAGCGCTCCTGGCGCTGC
TGGCTGCGCTCTGCCCGGCGAGTCGGGCTCTGGAGGAAAAGAAAGTTTGC
CAAGGCACGAGTAACAAGCTCACGCAGTTGGGCACTTTTGAAGATCATTT
TCTCAGCCTCCAGAGGATGTTCAATAACTGTGAGGTGGTCCTTGGGAATT
TGGAAATTACCTATGTGCAGAGGAATTATGATCTTTCCTTCTTAAAGACC
ATCCAGGAGGTGGCTGGTTATGTCCTCATGCTCTACAACCCCACCACGTA
CCAGATGGATGTGAACCCCGAGGGCAAATACACCTTTGGTGCCACCTGCG
TGAAGAAGTGTCCCCGTAATTATGTGGTGACAGATCACGGCTCGTGCGTC
CGAGCCTGTGGGGCCGACAGCTATGAGATGGAGGAAGACGGCGTCCGCAA
GTGTAAGAAGTGCGAAGGGCCTTGCCGCAAAGTGTGTAACGGAATAGGTA
TTGGTGAATTTAAAGACTCACTCTCCATAAATGCTACGAATATTAAACAC
TTCAAAAACTGCACCTCCATCAGTGGCGATCTCCACATCCTGCCGGTGGC
ATTTAGGGGTGACTCCTTCACACATACTCCTCCTCTGGATCCACAGGAAC
TGGATATTCTGAAAACCGTAAAGGAAATCACAGGGTTTTTGCTGATTCAG
GCTTGGCCTGAAAACAGGACGGACCTCCATGCCTTTGAGAACCTAGAAAT
CATACGCGGCAGGACCAAGCAACATGGTCAGTTTTCTCTTGCAGTCGTCA
GCCTGAACATAACATCCTTGGGATTACGCTCCCTCAAGGAGATAAGTGAT
GGAGATGTGATAATTTCAGGAAACAAAAATTTGTGCTATGCAAATACAAT
AAACTGGAAAAAACTGTTTGGGACCTCCGGTCAGAAAACCAAAATTATAA
GCAACAGAGGTGAAAACAGCTGCAAGGCCACAGGCCAGGTCTGCCATGCC
TTGTGCTCCCCCGAGGGCTGCTGGGGCCCGGAGCCCAGGGACTGCGTCTC
TTGCCGGAATGTCAGCCGAGGCAGGGAATGCGTGGACAAGTGCAACCTTC
TGGAGGGTGAGCCAAGGGAGTTTGTGGAGAACTCTGAGTGCATACAGTGC
CACCCAGAGTGCCTGCCTCAGGCCATGAACATCACCTGCACAGGACGGGG
ACCAGACAACTGTATCCAGTGTGCCCACTACATTGACGGCCCCCACTGCG
TCAAGACCTGCCCGGCAGGAGTCATGGGAGAAAACAACACCCTGGTCTGG
AAGTACGCAGACGCCGGCCATGTGTGCCACCTGTGCCATCCAAACTGCAC
CTACGGATGCACTGGGCCAGGTCTTGAAGGCTGTCCAACGAATGGGCCTA
AGATCCCGTCCATCGCCACTGGGATGGTGGGGGCCCTCCTCTTGCTGCTG
GTGGTGGCCCTGGGGATCGGCCTCTTCATGCGAAGGCGCCACATCGTTCG
GAAGCGCACGCTGCGGAGGCTGCTGCAGGAGAGGGAGCTTGTGGAGCCTC
TTACACCCAGTGGAGAAGCTCCCAACCAAGCTCTCTTGAGGATCTTGAAG
GAAACTGAATTCAAAAAGATCAAAGTGCTGGGCTCCGGTGCGTTCGGCAC
GGTGTATAAGGGACTCTGGATCCCAGAAGGTGAGAAAGTTAAAATTCCCG
TCGCTATCAAGGAATTAAGAGAAGCAACATCTCCGAAAGCCAACAAGGAA
ATCCTCGATGAAGCCTACGTGATGGCCAGCGTGGACAACCCCCACGTGTG
CCGCCTGCTGGGCATCTGCCTCACCTCCACCGTGCAGCTCATCACGCAGC
TCATGCCCTTCGGCTGCCTCCTGGACTATGTCCGGGAACACAAAGACAAT
ATTGGCTCCCAGTACCTGCTCAACTGGTGTGTGCAGATCGCAAAGGGCAT
GAACTACTTGGAGGACCGTCGCTTGGTGCACCGCGACCTGGCAGCCAGGA
ACGTACTGGTGAAAACACCGCAGCATGTCAAGATCACAGATTTTGGGCTG
GCCAAACTGCTGGGTGCGGAAGAGAAAGAATACCATGCAGAAGGAGGCAA
AGTGCCTATCAAGTGGATGGCATTGGAATCAATTTTACACAGAATCTATA
CCCACCAGAGTGATGTCTGGAGCTACGGGGTGACCGTTTGGGAGTTGATG
ACCTTTGGATCCAAGCCATATGACGGAATCCCTGCCAGCGAGATCTCCTC
CATCCTGGAGAAAGGAGAACGCCTCCCTCAGCCACCCATATGTACCATCG
ATGTCTACATGATCATGGTCAAGTGCTGGATGATAGACGCAGATAGTCGC
CCAAAGTTCCGTGAGTTGATCATCGAATTCTCCAAAATGGCCCGAGACCC
CCAGCGCTACCTTGTCATTCAGGGGGATGAAAGAATGCATTTGCCAAGTC
CTACAGACTCCAACTTCTACCGTGCCCTGATGGATGAAGAAGACATGGAC
GACGTGGTGGATGCCGACGAGTACCTCATCCCACAGCAGGGCTTCTTCAG
CAGCCCCTCCACGTCACGGACTCCCCTCCTGAGCTCTCTGAGTGCAACCA
GCAACAATTCCACCGTGGCTTGCATTGATAGAAATGGGCTGCAAAGCTGT
CCCATCAAGGAAGACAGCTTCTTGCAGCGATACAGCTCAGACCCCACAGG
CGCCTTGACTGAGGACAGCATAGACGACACCTTCCTCCCAGTGCCTGGTG
AGTGGCTTGTCTGGAAACAGTCCTGCTCCTCAACCTCCTCGACCCACTCA
GCAGCAGCCAGTCTCCAGTGTCCAAGCCAGGTGCTCCCTCCAGCATCTCC
AGAGGGGGAAACAGTGGCAGATTTGCAGACACAGTGAAGGGCGTAAGGAG
CAGATAAACACATGACCGAGCCTGCACAAGCTCTTTGTTGTGTCTGGTTG
TTTGCTGTACCTCTGTTGTAAGAATGAATCTGCAAAATTTCTAGCTTATG
AAGCAAATCACGGACATACACATCTGTATGTGTGAGTGTTCATGATGTGT
GTACATCTGTGTATGTGTGTGTGTGTATGTGTGTGTTTGTGACAGATTTG
ATCCCTGTTCTCTCTGCTGGCTCTATCTTGACCTGTGAAACGTATATTTA
ACTAATTAAATATTAGTTAATATTAATAAATTTTAAGCTTTATCCAGAAA
AAAAAAAAAAAAA

The present invention features a novel EGFR variant, EGFRvVI, comprising the nucleotide sequence containing a portion of EGFRvVI, specifically exons 1, 2, 3, 7 and 8 as follows, wherein the underlined sequence indicates the homologous region that constitutes the breakpoint within exons 3-7:

(SEQ ID NO: 4)
GCTGGCTGCGCTCTGCCCGGCGAGTCGGGCTCTGGAGGAAAAGAAAGTTT
GCCAAGGCACGAGTAACAAGCTCACGCAGTTGGGCACTTTTGAAGATCAT
TTTCTCAGCCTCCAGAGGATGTTCAATAACTGTGAGGTGGTCCTTGGGAA
TTTGGAAATTACCTATGTGCAGAGGAATTATGATCTTTCCTTCTTAAAGA
CCATCCAGGAGGTGGCTGGTTATGTCCTCATGCTCTACAACCCCACCACG
TACCAGATGGATGTGAACCCCGAGGGCAAATACACCTTTGGTGCCACCTG
CGTGAAGAAGTGTCCCCGTAATTATGTGGTGACAGATCACGGCTCGTGCG
TCCGAGCCTGTGGGGCCGACAGCTATGAGATGGAGGAA

The present invention features a novel EGFR variant, EGFRvVI, with the amino acid sequence as follows:

(SEQ ID NO: 5)
MRPSGTAGAALLALLAALCPASRALEEKKVCQGTSNKLTQLGTFEDHFLS
LQRMFNNCEVVLGNLEITYVQRNYDLSFLKTIQEVAGYVLMLYNPTTYQM
DVNPEGKYSFGATCVKKCPRNYVVTDHGSCVRACGADSYEMEEDGVRKCK
KCEGPCRKVCNGIGIGEFKDSLSINATNIKHFKNCTSISGDLHILPVAFR
GDSFTHTPPLDPQELDILKTVKEITGFLLIQAMPENRIDLHAFENLEIIR
GRTKQHGQFSLAVVSLNITSLGLRSLKEISDGDVIISGNKNLCYANTINW
KKLFGTSGQKTKIISNRGENSCKATGQVCHALCSPEGCWGPEPRDCVSCR
NVSRGRECVDKCNLLEGEPREFVENSECIQCHPECLPQAMNITCTGRGPD
NCIQCAHYIDGPHCVKTCPAGVMGENNTLVWKYADAGHVCHLCHPNCTYG
CTGPGLEGCPTNGPKIPSIATGMVGALLLLLVVALGIGLFMRRRHIVRKR
TLRRLLQERELVEPLTPSGEAPNQALLRILKETEFKKIKVLGSGAFGTVY
KGLWIPEGEKVKIPVAIKELREATSPKANKEILDEAYVMASVDNPHVCRL
LGICLTSTVQLITQLMPFGCLLDYVREHKDNIGSQYLLNWCVQIAKGMNY
LEDRRLVHRDLAARNVLVKTPQHVKITDFGLAKLLGAEEKEYHAEGGKVP
IKWMALESILHRIYTHQSDVWSYGVTVWELMTFGSKPYDGIPASEISSIL
EKGERLPQPPICTIDVYMIMVKCWMIDADSRPKFRELIIEFSKMARDPQR
YLVIQGDERMHLPSPTDSNFYRALMDEEDMDDVVDADEYLIPQQGFFSSP
STSRTPLLSSLSATSNNSTVACIDRNGLQSCPIKEDSFLQRYSSDPTGAL
TEDSIDDTFLPVPGEWLVWKQSCSSTSSTHSAAASLQCPSQVLPPASPEG
ETVADLQTQ

In some embodiments, EGFRvVI is preferentially expressed in tumor cells. Exemplary tumor cells include glioblastoma cells. Other tumor cells can include epithelial tumor cells. Tumor cells can include, but are not limited to tumor cells of the following cancers: glioblastoma, astrocytoma, meningioma, head and neck squamous cell cancer, melanoma, cervical cancer, renal cell cancer, lung cancer, prostate cancer, bladder cancer, colorectal cancer, pancreatic cancer and breast cancer.

Identification of EGFRvVI in a patient may be used to determine whether the patient has cancer, is at risk for developing cancer, or has a higher predisposition for developing cancer. This is due in part to the fact that EGFR variants are not expressed in healthy subjects that do not have cancer. In some embodiments, certain EGFR variants may be more prevalent in certain cancers. For example, EGFRvIII is the most frequent EGFR mutation found in non-small cell lung carcinoma (NSCLC) and GBM. In some embodiments, EGFRvVI is found in patients that have a neurologic cancer, for example, GBM or astrocytoma.

Cancer, as used herein, refers to a hyperproliferative condition or disorder. The term encompasses malignant as well as non-malignant cell populations. Such disorders are characterized by increased or excessive cell proliferation of one or more subsets of cells, which often appear to differ from the surrounding tissue both morphologically and genotypically. The increased or excessive cell proliferation in a tissue sample from a patient can be determined by comparison of cell proliferation from the tissue sample to a reference sample. The reference sample can be a score determined from a population of samples from a general population or a population of healthy subjects or subjects that have not been diagnosed with cancer. The reference sample can also be a sample from the same patient obtained at a different timepoint. Cell proliferation can be measured by any of the methods known in the art, such as immunohistochemical staining of biological samples for proliferation markers, such as Ki-67, BrDU, or mitotic markers. Hyperproliferative cell disorders can occur in different types of animals and in humans, and produce different physical manifestations depending upon the affected cells.

Cancers include neurologic cancers, epithelial cancers and solid tumor cancers. Such cancers include glioblastoma, astrocytoma, meningioma, head and neck squamous cell cancer, melanoma, cervical cancer, renal cell cancer, lung cancer, prostate cancer, bladder cancer, colorectal cancer, pancreatic cancer and breast cancer.

Cancers of particular interest are neurologic cancers, including brain tumors. Neurologic tumors are classified according to the kind of cell from which the tumor seems to originate, e.g., astrocytes. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astrocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by high proliferation indices, endothelial proliferation and focal necrosis. The highly proliferative nature of these lesions likely results from multiple mitogenic effects. One of the hallmarks of GBM is endothelial proliferation. A host of growth factors and their receptors are found amplified and/or mutated in GBMs.

### Antibodies

The EGFR variant of the present invention, including fragments, derivatives and analogs thereof, may be used as an immunogen to produce antibodies having use in the diagnostic, research, and therapeutic methods described below. The antibodies may be polyclonal or monoclonal, chimeric, humanized, single chain (svFc) or Fab fragments. Various procedures known to those of ordinary skill in the art may be used for the production and labeling of such antibodies and fragments. See, e.g., Burns, ed., Immunochemical Protocols, 3.sup.rd ed., Humana Press (2005); Harlow and Lane, Antibodies: A Laboratory Manual, Cold Spring Harbor Laboratory (1988); Kozbor et al., Immunology Today 4: 72 (1983); Kohler and Milstein, Nature 256: 495 (1975).

Antibodies or fragments exploiting the differences between EGFRvVI and full length wild-type EGFR or other EGFR variants are particularly preferred. Of particular interest are antibodies or functional fragments thereof that specifically recognize EGFRvVI. What is meant by “specifically recognize” is that the antibodies or functional fragments thereof preferentially bind to EGFRvVI compared to wild-type EGFR and/or other EGFR variants, for example, EGFRvIII. For example, the antibody may bind to the region of EGFRvVI in which exons 3 and 7 are fused. Alternatively, the three-dimensional, or native, conformation of the native EGFRvVI protein may present a novel epitope that may not exist or may not accessible in wild-type EGFR or other EGFR variants, which can be recognized by the EGFRvVI-specific antibodies disclosed herein.

Exemplary uses for the EGFRvVI antibody are disclosed herein. Such uses include diagnostic and prognostic methods.

### Diagnostic Applications

The present invention provides DNA, RNA and protein based diagnostic methods that either directly or indirectly detect EGFRvVI. The present invention also provides compositions and kits for diagnostic purposes.

The present invention features a diagnostic method comprising obtaining a biological sample from a patient, extracting nucleic acids or proteins, and determining the presence, absence, or level of EGFRvVI in the biological sample. Methods for determining the presence, absence, or level of EGFRvVI in a biological sample are discussed further herein.

The diagnostic methods of the present invention may be qualitative or quantitative. Quantitative diagnostic methods may be used, for example, to discriminate between indolent and aggressive cancers via a predetermined cutoff or threshold level. Where applicable, qualitative or quantitative diagnostic methods may also include amplification of target, signal or intermediary (e.g., a universal primer).

EGFRvVI may be detected along with other markers in a multiplex or panel format. Markers are selected for their predictive value alone or in combination with the EGFR variant. Such markers may also include one or more of the other EGFR variants, such as EGFRvIII. Markers for other cancers, diseases, infections, and metabolic conditions known in the art are also contemplated for inclusion in a multiplex of panel format.

The diagnostic methods of the present invention may also include consideration to data correlating particular EGFR variants, such as EGFRvVI, with the stage, aggressiveness or progression of the disease or the presence or risk of metastasis. Ultimately, the information provided by the methods of the present invention will assist a physician in choosing the best course of treatment for a particular patient.

Samples

As used herein, the term “biological sample” refers to a sample that contains biological materials such as a DNA, an RNA and a protein. In some embodiments, the biological sample may be a tissue sample, a cell culture sample, or a biopsy sample. In some embodiments, the biological sample may suitably comprise a bodily fluid from a subject. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. In some embodiments, the preferred body fluid for use as the biological sample is urine. In other embodiments, the preferred body fluid is cerebrospinal fluid (CSF), serum or plasma. Suitably, a sample volume of about 0.1 ml to about 30 ml fluid may be used. The volume of fluid may depend on a few factors, e.g., the type of fluid used. For example, the volume of serum samples may be about 0.1 ml to about 20 ml, preferably about 4 ml. The volume of urine samples may be about 10 ml to about 30 ml, preferably about 20 ml.

In other embodiments, the sample may be in vivo, in which circulating tumor cells or microvesicles (e.g., exosomes) that express EGFRvVI can be detected from biofluids in vivo via insertion of a venous catheter. Specifically, the venous catheter comprises a detection agent that can bind to EGFRvVI, e.g., an EGFRvVI-specific antibody. The catheter then detects or collects the EGFRvVI-expressing circulating tumor cells or microvesicles. The venous catheter can be left inserted into the subject for at least 5 minutes, to survey at least 2.5 liters of blood. Thus, this method allows for detection of rare tumor events.

The term “subject” is intended to include all animals shown to or expected to express EGFR and variants thereof. In particular embodiments, the subject is a mammal, a human or nonhuman primate, a dog, a cat, a horse, a cow, other farm animals, or a rodent (e.g. mice, rats, guinea pig. etc.). A human subject may be a normal human being without observable abnormalities, e.g., a disease. A human subject may be a human being with observable abnormalities, e.g., a disease. The observable abnormalities may be observed by the human being himself, or by a medical professional. The term “subject”, “patient”, and “individual” are used interchangeably herein.

The biological sample may require preliminary processing designed to isolate or enrich the sample for EGFRvVI or other markers of interest. In some aspects, cells or other nucleic acid-containing particles that contain EGFRvVI or other markers of interest are isolated. A variety of techniques known to those of ordinary skill in the art may be used for this purpose, including but not limited: centrifugation; immunocapture; cell lysis; and nucleic acid target capture (See, e.g., EP Pat. No. 1 409 727, herein incorporated by reference in its entirety).

In some embodiments, it may be advantageous to pre-process the biological sample such that a microvesicle fraction is isolated, purified or enriched. As used herein, the term “microvesicles” refers collectively to all membrane vesicles less than 0.8 microns that are shed by a cell. Isolation of microvesicles from a biological sample can be achieved through centrifugation, filtration, ion exchange chromatography, size exclusion chromatography and affinity chromatography, or any combination thereof (See, e.g., PCT Applications WO2011/009104, WO2012/051622, and WO2012/064993). Isolation of the microvesicle fraction prior to extraction of nucleic acids or proteins results in higher quality extractions for more accurate or specific detection of EGFRvVI and other markers of interest.

Nucleic Acid Detection

EGFRvVI may be detected as chromosomal rearrangements of genomic DNA or chimeric mRNA using a variety of nucleic acid techniques known to those of ordinary skill in the art, including but not limited to: nucleic acid sequencing; nucleic acid hybridization; and nucleic acid amplification.

Examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack, RNA is usually reverse transcribed to DNA before sequencing. Methods and reagents for reverse-transcribing RNA to cDNA are known in the art. Chain terminator sequencing uses sequence-specific termination of a DNA synthesis reaction using modified nucleotide substrates, radioactive or other labeled oligonucleotide primers, and chain terminating nucleotides. Dye terminator sequencing labels the terminators and complete sequencing can be performed in a single reaction by labeling each di-deoxynucleotide chain terminator with a separate fluorescent dye.

Examples of nucleic acid hybridization techniques include, but are not limited to, in situ hybridization (ISH, such as fluorescent in situ hybridization FISH), microarray, and Southern or Northern blot. ISH uses a labeled complementary DNA or RNA probes to localize, or hybridize, to a specific DNA or RNA sequence in a portion or section of tissue (in situ). Examples of microarrays include DNA microarrays (e.g., cDNA microarrays and oligonucleotide microarrays), protein microarrays; tissue microarrays, transfection or cell microarrays, chemical compound microarrays, and antibody microarrays. A DNA microarray is also commonly known as a gene chip, DNA chip, or biochip. Microarrays are a collection of probes (e.g., DNA, RNA, compounds or antibodies) attached to a solid surface (e.g., glass, plastic or silicon chip) forming an array for the purpose of monitoring expression profiles of thousands of genes or proteins simultaneously. Southern blotting is utilized to detect specific filter-bound DNA sequences by hybridization with a labeled probe complementary to the sequence of interest. Northern blotting is utilized to detect specific filter-bound RNA sequences by hybridization with a labeled probe complementary to the sequence of interest.

EGFRvVI can be identified by amplification which is performed either prior to or simultaneously with detection. Examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety. Transcription-mediated amplification, commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. The ligase chain reaction, commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded and ligated oligonucleotide product. Strand displacement amplification, commonly referred to as SDA, uses cycles of annealing pairs of primer sequences to opposite strands of a target sequence, primer extension in the presence of dNTPαS to produce a duplex hemiphosphorothioated primer extension product. This primer extension product undergoes endonuclease-mediated nicking at a hemimodified restriction endonuclease recognition site, and polymerase-mediated primer extension from the 3′ end of the nick displaces an existing strand and produces a strand for the next round of primer annealing, nicking and strand displacement, thereby resulting in geometric amplification of product.

Non-amplified or amplified EGFR variant nucleic acids can be detected by any conventional means. For example, EGFRvVI can be detected by hybridization with a detectably labeled probe and measurement of the resulting hybrids. Illustrative non-limiting examples of detection methods are described below.

Quantitative evaluation of the amplification process can be performed in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

The present invention also provides nucleic acids that bind to EGFRvVI nucleic acids for the specific detection of the presence of EGFRvVI nucleic acid in a sample. The nucleic acids for detection of EGFRvVI comprise an isolated nucleic acid consisting of 10 to 1000 nucleotides (preferably, 10 to 500, 10 to 100, 10 to 50, 10 to 35, 20 to 1000, 20 to 500, 20 to 100, 20 to 50, or 20 to 35 nucleotides) which hybridizes to RNA or DNA encoding EGFRvVI or to an EGFR gene. In a preferred embodiment, the nucleic acids preferentially hybridize to RNA or DNA encoding EGFRvVI but not to RNA or DNA of wild-type EGFRvVI or other EGFR variants. Specifically, the isolated nucleic acid is or is complementary to a nucleotide sequence consisting of at least 10 consecutive nucleotides (preferably, 15, 18, 20, 25, or 30) from the nucleic acid molecule comprising a polynucleotide sequence at least 60%, 65%, 70%, 75%, 80%, 85%, 90%, 91%, 92%, 93%, 94%, 95%, 96%, 97%, 98%, or 99% identical to a sequence selected from the group consisting of: a nucleotide sequence encoding the EGFRvVI polypeptide comprising the amino acid sequence in SEQ ID NO:5; a nucleotide sequence comprising the nucleic acid sequence in SEQ ID NO: 3 or SEQ ID NO: 4; a nucleotide sequence of any one of exons 1-3, or exons 7-28 of EGFRvVI (SEQ ID NO:3) or wild-type EGFR, e.g., SEQ ID NO: 1, wherein the nucleotide sequence may also span adjacent exons; or a sequence complementary to any of the nucleotide sequences described above. Preferably, the nucleotide sequence does not comprise the sequence of sequence complementary to exon 4, exon 5, or exon 6 of wild-type EGFR, e.g., SEQ ID NO: 1. Complementary sequences are also known as antisense nucleic acids when they comprise sequences which are complementary to the coding strand. Preferably, the nucleic acid does not specifically hybridize to nucleotides that encode amino acids at positions 90-221 of wild-type EGFR. The nucleic acids useful for detection are also referred to herein as primers or probes and can be utilized in many of the detection methods described herein.

In one aspect, the isolated nucleic acid molecule for detection of EGFRvVI comprises 10 to 50 nucleotides, which specifically hybridize to the EGFRvVI RNA, DNA or cDNA. The nucleic acid molecule is, or is complementary to, a nucleotide sequence consisting of at least 5, at least 10, at least 15, or at least 20 consecutive nucleotides from EGFRvVI exons 1-3 or exons 7-28 of EGFRvVI (SEQ ID NO: 3) or wild-type EGFR (SEQ ID NO: 1), including nucleotide sequences that span adjacent exons of exons 1-3 and 7-28. Specifically, the nucleic acid molecules do not hybridize to the nucleotide sequences that encode amino acids 90-221 of wild-type EGFR.

Examples of specific nucleic acid primers and probes which can be used in the present invention include:

EGFR Forw 1-
(SEQ ID NO: 6)
CTGCTGGCTGCGCTCTG
EGFRv3 rev4-
(SEQ ID NO: 7)
CGTGATCTGTCACCACATAATTACC
EGFR probe 6-
(SEQ ID NO: 8)
TTCCTCCAGAGCCCGACT
EGFR Forw 1-
(SEQ ID NO: 6)
CTGCTGGCTGCGCTCTG
EGFR Rev 1-
(SEQ ID NO: 9)
TTCCTCCATCTCATAGCTGTCG
EGFR probe 6:
(SEQ ID NO: 8)
TTCCTCCAGAGCCCGACT
EGFR qPCR exon 3-7 del forw 1:
(SEQ ID NO: 10)
TGGTCCTTGGGAATTTGGAA
EGFR qPCR exon 3-7 del rev:
(SEQ ID NO: 11)
GTGGGGTTGTAGAGCATGAGGA
EGFR qPCR exon 3-7 del probe:
(SEQ ID NO: 12)
TACCTATGTGCAGAGGAA

As will be understood by the person of ordinary skill, a multitude of additional probes or primers can be designed from SEQ ID NO: 3.

The nucleic acid probe can be used to probe an appropriate chromosomal or cDNA library by usual hybridization methods to obtain another nucleic acid molecule of the present invention. A chromosomal DNA or cDNA library can be prepared from appropriate cells according to recognized methods in the art (cf. Molecular Cloning: A Laboratory Manual, second edition, edited by Sambrook, Fritsch, & Maniatis, Cold Spring Harbor Laboratory, 1989).

In the alternative, chemical synthesis is carried out in order to obtain nucleic acid probes having nucleotide sequences which correspond to N-terminal and C-terminal portions of the PCA3 amino acid sequence. Thus, the synthesized nucleic acid probes can be used as primers in a polymerase chain reaction (PCR) carried out in accordance with recognized PCR techniques, essentially according to PCR Protocols, A Guide to Methods and Applications, edited by Michael et al., Academic Press, 1990, utilizing the appropriate chromosomal, cDNA or cell line library to obtain the fragment of the present invention.

One skilled in the art can readily design such probes based on the sequence disclosed herein using methods of computer alignment and sequence analysis known in the art (cf. Molecular Cloning: A Laboratory Manual, second edition, edited by Sambrook, Fritsch, & Maniatis, Cold Spring Harbor Laboratory, 1989).

The hybridization probes of the present invention can be labeled by standard labeling techniques such as with a radiolabel, enzyme label, fluorescent label, biotin-avidin label, chemiluminescence, and the like. For example, the probe is labeled with FAM dye. After hybridization, the probes can be visualized using known methods.

The nucleic acid primers and probes of the present invention include RNA, as well as DNA probes, such probes being generated using techniques known in the art.

In one embodiment of the above described method, a nucleic acid probe is immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

Protein Detection

EGFRvVI of the present invention may be detected as truncated a EGFR protein, a chimeric protein or a protein with a deletion using a variety of protein techniques known to those of ordinary skill in the art, including but not limited to protein sequencing and immunoassays. Examples of protein sequencing techniques include, but are not limited to, mass spectrometry and Edman degradation. Examples of immunoassays include, but are not limited to immunoprecipitation, Western blot, ELISA, immunohistochemistry, immunocytochemistry, flow cytometry, and immuno-PCR. Polyclonal or monoclonal antibodies labeled with detectable moieties using various techniques known to those of ordinary skill in the art (e.g., calorimetric, fluorescent, chemiluminescent or radioactive labels) are suitable for use in the immunoassays.

In some embodiments, a computer-based analysis program is used to translate the raw data generated by the detection assay (e.g., the presence, absence, or amount of a given marker or markers, for example EGFRvVI) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject. The present invention contemplates any method capable of receiving, processing, and transmitting the information to and from laboratories conducting the assays, information provides, medical personal, and subjects.

EGFRvVI may also be detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence of or expression of cancer markers in an animal (e.g., a human or nonhuman mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. The in vivo imaging methods of the present invention are useful in the diagnosis of cancers that express the cancer markers of the present invention (e.g., glioblastoma). In vivo imaging is used to visualize the presence of a marker indicative of the cancer, e.g., EGFRvVI. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

Compositions & Kits

Compositions for use in the diagnostic methods of the present invention include, but are not limited to, probes, amplification oligonucleotides, and antibodies. Particularly preferred compositions detect EGFRvVI, but not any other EGFR variant. These compositions include: a single labeled probe or oligonucleotide primer comprising a sequence that hybridizes to the junction at which a 5′ portion from exon 3 of EGFR fuses to a 3′ portion from exon 7; a pair of amplification oligonucleotides wherein the one amplification oligonucleotide comprises a sequence that hybridizes to the homologous breakpoint where exon 3 and exon 7 is joined in EGFRvVI; or an antibody that specifically recognizes EGFRvVI.

Any of these compositions, alone or in combination with other compositions of the present invention, may be provided in the form of a kit. For example, the single labeled probe and pair of amplification oligonucleotides may be provided in a kit for the amplification and detection of the EGFR variant of the present invention. Kits may further comprise appropriate controls and/or detection reagents.

The probe and antibody compositions of the present invention may also be provided in the form of an array.

### Prognostic Applications

A correlation between EGFRvVI and the prognosis of patients with cancer may exist. In some embodiments, the presence, absence or level of the EGFRvVI may be of use for stratifying or classifying the tumor based on its activity, aggressiveness, invasiveness, or metastatic potential. Thus, in some embodiments, assays for detecting EGFRvVI are used to provide prognoses and help physicians decide on an appropriate therapeutic strategy. For example, in some embodiments, patients with tumors that have EGFRvVI are treated differently than those that have wild-type EGFR. For example, the prognosis of patients that have EGFRvVI may be worse than patients that lack EGFRvVI, and therefore may require more aggressive treatment regimens. In some embodiments, the presence of EGFRvVI correlates with survival rate/time, invasiveness of the tumor, and metastasis.

Although the present invention will most preferably be used in connection with obtaining a prognosis for glioblastoma cancer patients, other epithelial or solid cell tumors may also be examined and the assays and probes described herein may be used in determining whether cancerous cells from these tumors express EGFRvVI, which is likely to make them particularly aggressive, i.e., likely to be invasive and metastatic. Examples of tumors that may be characterized using this procedure include tumors of the breast, lung, colon, ovary, uterus, esophagus, stomach, liver, kidney, brain, skin and muscle. The assays of the present invention will also be of value to researchers studying these cancers in cell lines and animal models.

### Drug-Screening and Therapeutic Applications

In some embodiments, the present invention provides drug-screening assays (e.g., to screen for anticancer drugs). The screening methods of the present invention provide for identification of compounds or agents (e.g., small molecules, drugs, proteins, peptides, peptidomimetics, peptoids) that can modulate the biological function of EGFRvVI. For example, the compounds or agents can inhibit EGFRvVI in a ligand-independent manner, as EGFRvVI lacks a portion of the ligand-binding domain. Inhibiting the biological function of EGFRvVI can include inhibiting the EGFR kinase activity, modulating signaling pathways that are upstream or downstream of EGFRvVI, or increasing proteasomal degradation of EGFRvVI. Compounds that inhibit the activity of EGFRvVI may be useful in the treatment of proliferative disorders, e.g., cancer.

In some embodiments, it may be advantageous to modulate or inhibit EGFRvVI for treatment of proliferative disorders, such as cancer. The present invention encompasses therapies that target EGFRvVI directly or indirectly. Examples of therapies useful for inhibiting the function of EGFRvVI include RNA interference (RNAi) compounds such as siRNAs, and other antisense compounds that specifically hybridize to target nucleic acids encoding EGFRvVI. Other therapies that may be useful include antibodies that specifically bind and/or inhibit activity of EGFRvVI. In some embodiments, the antibody may be conjugated to a cytotoxic agent (e.g., toxin, drug or radioactive moiety).

In some embodiments, the present invention features methods for detecting EGFRvVI to determine or assess the responsiveness of a subject to a particular therapeutic regimen comprising analyzing the presence or absence of EGFRvVI wherein the presence or absence correlates with the responsiveness of a subject to the therapeutic regiment. The methods of detection can be used to detect EGFRvVI DNA, RNA or protein, as described herein. Preferably, the method of detection is by nucleic acid amplification, e.g., quantitative real-time PCR.

EGFR has been a popular target for molecular therapies for cancer or proliferative disorders. EGFR-specific therapies generally include inhibitors of the EGFR kinase activity and inhibitors of the ligand-mediated activation of EGFR. Examples of therapies that target EGFR include antibody inhibitors that block the extracellular N-terminal ligand binding domain. Blocking of the ligand binding domain prevents inappropriate activation and downstream signaling of EGFR. Examples of agents that block the ligand-binding domain of EGFR include cetuximab (ERBITUX®), panitumumab (VECTIBIX®), zalutumumab, nimotuzumab (BioMab EGFR), and matuzumab (EMD 7200). Other EGFR-specific inhibitors include small molecules that inhibit the EGFR tyrosine kinase activity by reversibly or irreversibly binding the ATP-binding pocket. These small molecules target the kinase domain, which is on the cytoplasmic and C-terminal end of the protein. Such inhibitors include, for example, gefitinib (IRESSA™), erlotinib (TARCEVA®) and lapatinib (TYKERB®). Other EGFR inhibitors include cancer vaccines that target EGFR deletions, such as Rindopepimut (CDX-110, Celldex Therapeutics). Other inhibitors that may be particularly useful for indirectly inhibiting EGFR and downstream signaling is PARP1 inhibitors, for example, 5-AIQ hydrochloride, ABT-888, minocycline, PARP inhibitor IX (EB-47), PARP inhibitor XI (DR2313), TIQ-A (available from Santa Cruz Biotechnology).

However, not all patients respond to treatments with these EGFR inhibitors, which can be partly attributable to the tumor-specific EGFR variants that exhibit deletions of the portions of EGFR that are targeted by certain therapeutic agents. For example, EGFRvVI lacks a significant portion of the N-terminal ligand binding domain. Presence of EGFRvVI may indicate that therapeutic regimens comprising molecules that target the ligand binding domain will not be effective in treatment of cancer. Presence of EGFRvVI may indicate that the subject will not be responsive to therapeutic regimens comprising molecules that target the ligand binding domain. Accordingly, detection of the presence of EGFRvVI may direct those skilled in the art to rule out treatment regimens that comprise EGFR inhibitors.

### EXAMPLES

### Example 1: Identification of Novel EGFR Variant

In this example, a glioma cerebrospinal fluid (CSF) sample was obtained from a patient (Patient ID UCS-0001). The biopsy was EGFRvIII positive. Microvesicles were extracted from 4 ml of CSF sample. Nucleic acids, for example RNA, were extracted by methods known in the art.

Two EGFR PCR-based detection assays were performed. In the EGFRvIII-specific assay, the following primers and probe were used:

EGFR Forw 1-
(SEQ ID NO: 6)
CTGCTGGCTGCGCTCTG
EGFRv3 rev4-
(SEQ ID NO: 7)
CGTGATCTGTCACCACATAATTACC
EGFR probe 6-
(SEQ ID NO: 8)
TTCCTCCAGAGCCCGACT (FAM-dye labeled Minor Grove
Binder (MGB) probe)

In the second EGFR PCR-based detection assay, the following primers and probe were used:

EGFR Forw 1-
(SEQ ID NO: 6)
CTGCTGGCTGCGCTCTG
EGFR Rev 1-
(SEQ ID NO: 9)
TTCCTCCATCTCATAGCTGTCG
EGFR probe 6-
(SEQ ID NO: 8)
TTCCTCCAGAGCCCGACT (FAM-dye labeled Minor Grove
Binder (MGB) probe)

The EGFRvIII specific assay (EGFR Forw 1, EGFRv3rev4, EGFR probe 6) did not produce an amplification product from the CSF sample. However, the second EGFR assay (EGFR Forw 1, EGFR Rev 1 and EGFR probe 6) produced an amplification product.

This amplification product from the second EGFR assay was further investigated by a qualitative PCR amplification. The following PCR setup was used:

| | |
|---|---|
| EGFR Forw 1 (18 uM) | 1 ul |
| EGFR Rev 1 (18 uM) | 1 ul |
| dNTPs (10 mM) | 1 ul |
| HF enzyme buffer (5×) | 10 ul |
| Phusion hot start II DNA polymerase | 0.5 ul |
| DMSO | 1.5 ul |
| H2O | 30 ul |
| cDNA | 5 ul |

The PCR cycling conditions were as follows:

1. 98° C., 30 sec

2. 98° C., 10 sec

3. 62° C., 15 sec

4. 72° C., 15 sec

5. 72° C., 5 min

Repeat step 2-4 for 36 cycles.

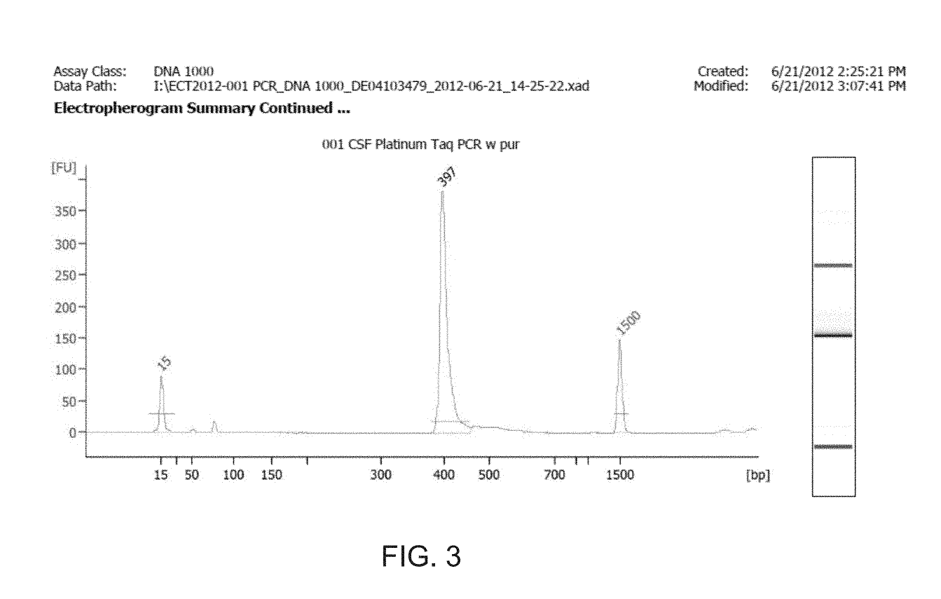

The amplified PCR product from the EGFR assay showed a larger band than EGFRvIII when analyzed. Bioanalyzer results were generated into an electropherogram, as shown in FIG. 3. The amplified PCR product was cloned into a TOPO vector and sequenced. The nucleotide sequence of exons 1, 2, part of 3, part of 7 and 8 is shown in FIG. 4.

Sequencing of the amplified PCR product identified a break a third into exon 3 that joins halfway into exon 7. EGFRvIII is a deletion in which the whole exon 2-7 is deleted, wherein the DNA break can be anywhere in the intronic regions. In contrast, the newly identified EGFR variant is characterized by a DNA break that occurs within the exons, and thus, has a defined junction on DNA. EGFRvVI contains a deletion that is 10730 bp long, and includes coding and non-coding regions. EGFRvVI nucleotide sequence encodes a polypeptide that lacks amino acids 90-221 of the WT EGFR, as shown in FIG. 5.

Additional examples of primers that can be used for amplification of EGFRvVI in addition to EGFR Forw1 (SEQ ID NO: 6) and EGFR Rev1 (SEQ ID NO: 9) are as follows:

EGFR qPCR exon 3-7 del forw 1:
(SEQ ID NO: 10)
TGGTCCTTGGGAATTTGGAA
EGFR qPCR exon 3-7 del rev:
(SEQ ID NO: 11)
GTGGGGTTGTAGAGCATGAGGA
EGFR qPCR exon 3-7 del probe (FAM-MGB):
(SEQ ID NO: 12)
TACCTATGTGCAGAGGAA

While the present invention has been disclosed with reference to certain embodiments, numerous modifications, alterations, and changes to the described embodiments are possible without departing from the full scope of the invention, as described in the appended specification and claims.

What is claimed is:

1. An isolated epithelial growth factor receptor (EGFR) protein comprising an in-frame deletion that starts in exon 3 and ends in exon 7 of wild-type EGFR.

2. The isolated epithelial growth factor receptor (EGFR) protein of claim 1, wherein the amino acids 90-221 have been deleted.

3. The isolated epithelial growth factor receptor (EGFR) protein of claim 1, wherein said EGFR protein comprises the amino acid sequence of SEQ ID NO: 5.

4. An isolated polynucleotide coding for the epithelial growth factor receptor (EGFR), wherein the nucleotides coding for amino acids 90-221 are deleted.

5. An isolated polynucleotide coding for the epithelial growth factor receptor (EGFR), wherein said polynucleotide comprises the nucleotide sequence of SEQ ID NO: 3 or 4.

6. An antibody that binds to the EGFR protein of claim 1.

7. A method for diagnosing cancer in a subject comprising analyzing a biological sample for the presence of the EGFR protein of claim 1 or a polynucleotide encoding the EGFR protein of claim 1, wherein presence of the EGFR protein or polynucleotide indicates the presence of a cancer or a higher predisposition of the subject to develop a cancer.

8. The method of claim 7, wherein the cancer is a neurologic cancer or an epithelial cancer.

9. The method of claim 8, wherein the neurologic cancer is glioblastoma or glioblastoma multiforme.

10. The method of claim 8, wherein the epithelial cancer is colon cancer, lung cancer, prostate cancer, breast cancer or other solid tumor cancers.

11. A method for assessing the responsiveness of a subject to a therapeutic regimen comprising providing a biological sample from a subject and determining the presence, absence, or level of expression of the EGFR protein of claim 1 or a polynucleotide encoding the EGFR protein of claim 1, wherein the presence, absence, or level of expression of the EGFR protein or polynucleotide indicates the responsiveness of the subject to the therapeutic regimen.

12. The method of claim 11, wherein the therapeutic regimen comprises an EGFR inhibitor.

13. A method for recommending a therapeutic regimen comprising providing a biological sample from a subject, determining the presence of the EGFR protein of claim 1 or a polynucleotide encoding the EGFR protein of claim 1, wherein the presence of the EGFR protein or polynucleotide rules out a therapeutic regimen comprising an EGFR inhibitor.

14. The method of claim 12, wherein the EGFR inhibitor is a small molecule, RNAi agent, antibody, or drug that inhibits EGFR expression or activation.

15. A method for targeting a cancer cell that expresses the EGFR protein of claim 1 comprising administering to a subject an agent that specifically binds to the EGFR protein.

16. The method of claim 15, wherein said agent is an antibody.

17. The method of claim 15, wherein said antibody is covalently linked to a therapeutic agent.

18. The method of claim 17, wherein said therapeutic agent is a chemotherapeutic agent, an anti-cancer agent, a toxin, a radioisotope, or a nanoparticle.

19. A method for treating a cancer or a symptom thereof, comprising administering to a subject an inhibitor of the EGFR of claim 1.

20. The method of claim 19, wherein said inhibitor is a small molecule, RNAi agent, antibody, or drug that inhibits the expression or activation of the EGFR of claim 1.


SEQUENCE LISTING (14 sequences)

SEQ ID NO: 1 - DNA, 3859 bp, Homo sapiens
gtccgggcagcccccggcgcagcgcggccgcagcagcctcctccccccgcacggtgtgagcgcccgccgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggacaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtatgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccagaaaaaaaaaaaaaaaa

SEQ ID NO: 2 - protein, 1091 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 3 - DNA, 3463 bp, Homo sapiens
gtccgggcagcccccggcgcagcgcggccgcagcagcctcctccccccgcacggtgtgagcgcccgccgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcatgctctacaaccccaccacgtaccagatggatgtgaaccccgagggcaaatacacctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtatgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccagaaaaaaaaaaaaaaaa

SEQ ID NO: 4 - DNA, 388 bp, Homo sapiens
gctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcatgctctacaaccccaccacgtaccagatggatgtgaaccccgagggcaaatacacctttggtgccacctgcgtgaagaagtgtccccgtaattatgtggtgacagatcacggctcgtgcgtccgagcctgtggggccgacagctatgagatggaggaa

SEQ ID NO: 5 - protein, 959 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 6 - DNA, 17 bp, Artificial Sequence
  Chemically synthesized primer
ctgctggctgcgctctg

SEQ ID NO: 7 - DNA, 25 bp, Artificial Sequence
  Chemically synthesized primer
cgtgatctgtcaccacataattacc

SEQ ID NO: 8 - DNA, 18 bp, Artificial Sequence
  Chemically synthesized primer
ttcctccagagcccgact

SEQ ID NO: 9 - DNA, 22 bp, Artificial Sequence
  Chemically synthesized primer
ttcctccatctcatagctgtcg

SEQ ID NO: 10 - DNA, 20 bp, Artificial Sequence
  Chemically synthesized primer
tggtccttgggaatttggaa

SEQ ID NO: 11 - DNA, 22 bp, Artificial Sequence
  Chemically synthesized primer
gtggggttgtagagcatgagga

SEQ ID NO: 12 - DNA, 18 bp, Artificial Sequence
  Chemically synthesized primer
tacctatgtgcagaggaa

SEQ ID NO: 13 - skipped

SEQ ID NO: 14 - DNA, 388 bp, Homo sapiens; identical to SEQ ID NO: 4